(19) United States
(12) Patent Application Publication
(10) Pub. No.: US 2021/0106762 A1
(43) Pub. Date: Apr. 15, 2021

(54) PREFILLABLE SAFETY SYRINGE

(71) Applicants: Menino Osbert Cotta, Wheelers Hill (AU); Jose Olavo Cotta, Wheelers Hill (AU); Largos Services Pty Ltd, Wheelers Hill (AU)

(72) Inventors: Menino Osbert Cotta, Wheelers Hill (AU); Jose Olavo Cotta, Wheelers Hill (AU)

(21) Appl. No.: 17/047,759

(22) PCT Filed: Mar. 7, 2019
(86) PCT No.: PCT/AU2019/050199

§ 371 Date: Oct. 15, 2020

(30) Foreign Application Priority Data
Apr. 26, 2018 (IN) 201821015743

(51) Int. Cl. A61M 5/315; A61M 5/32; A61M 5/31; A61M 5/34

(52) U.S. Cl. CPC A61M 5/3157; A61M 5/3204; A61M 2005/3126; A61M 5/34; A61M 2205/583; A61M 5/3137

(57) ABSTRACT

The present invention relates to a prefillable syringe with safety features that renders it auto-disable with user-controlled needle retraction after use. These features prevent re-use and greatly minimise the risk of needle stick injuries. The prefillable syringe has simple mechanisms that are devoid of any spring components and enables efficient administration of medicine without any possibility of leakage. The autodisable mechanism occurs via a locking arrangement between the needle holder and plunger, while needle retraction is facilitated via a locking arrangement between the plunger and plunger extension. Tamper prevention of the auto-disable mechanism occurs through a uniquely designed finger flange extension and/or barrel design modification. The unique design of the barrel tip allows for the needle cap to securely form a seal over the needle (if staked) to prevent any leakage prior to use.

### FIELD OF INVENTION

The present invention generally relates to a prefillable syringe with safety features which renders it auto-disable with retractable needle after intended use.

### BACKGROUND OF THE INVENTION

The administration of therapeutics via injection is an indispensable delivery route for numerous medicines and biologicals critical to patient health and well-being. Prefillable syringes are gaining increased popularity due to their many demonstrated advantages including greater medication safety, reduced loss of drug substance and increased convenience compared to a conventional stoppered vial and syringe. In addition, the medication errors are greatly reduced because the drug is easily identifiable at the point of use.

The availability of an increasing number of these therapeutics in prefillable insertable and disposable cartridges are also propelling the growth of prefillable syringes. Moreover, the cost of expensive biologicals, such as recombinant proteins, has created interest in prefillable syringes to reduce cost and waste associated with the handling of vial-packaged therapeutics.

Needle stick injuries, also referred to as sharps injuries, are among the most common injuries to healthcare professionals and patients all over the world, and therefore injection safety is a high priority when developing prefillable syringes.

Healthcare workers may experience a sharps injury while administering a vaccine or a curative injection, inadvertently recapping a needle, disassembling a syringe or during a process of handling and disposing of medical waste. In its 2002 World Health Report, the World Health Organisation (WHO) reported that of the 35 million health workers in the world, two million experience a percutaneous exposure to infectious diseases every year. The WHO regional report suggests that, on average, a health worker receives four sharps injuries per year. The scale of the problem has prompted some governments to respond with policies to protect healthcare workers from this occupational hazard. The WHO recommends the use of syringes with a sharps injury protection feature (SIP devices) for health care workers delivering intramuscular, subcutaneous or intradermal injectable medications to patients.

Hence, preventing accidental injury and infection from used hypodermic needles has attracted considerable interest in recent years and has created a need for technological advances in medical equipment.

In order to minimize the risk of needle stick injuries, retractable syringes have been developed which enable retraction of the needle into the barrel of the syringe following use, and prior to disposal. Retraction and retention of the used needle into the barrel protectively isolates the needle and prevents further human contact.

Re-use of syringes is endemic in developing countries and has led to the development of auto-disable syringes. The WHO and its partners recommend the use of auto-disable syringes, “bundled” with the supply of vaccines in some circumstances, and in all routine immunization campaigns.

As an example, the U.S. Pat. No. 7,988,675, titled, “Automatic injection and retraction devices for use with pre-filled syringe cartridges” describes an automatic injection and retraction device. The retraction and injection assemblies are configured so that, upon application of a twisting torque to the injection and retraction assemblies, the activation-prevention feature moves from an on position to the off position simultaneous with applying the removal force to the needle guard.

The syringe described above has workable mechanisms to retract the needle so that any untoward exposure to the contaminated needle and or the syringes can be successfully avoided. However, most of these syringes which retract the needle into the barrel of the syringe have inconvenient, complicated locking or disengagement steps and multiple components including spring mechanisms to release the needle from its injecting position so as to allow it to be withdrawn into the barrel. Because of the complexities of the retracting mechanism and the technology involved, auto-disable, retractable syringes tend to become complicated and costly as compared to conventional syringes.

Accordingly, there is a need to simplify such mechanisms in order to reduce cost, providing a viable, low-cost alternative to existing syringes in the market.

### OBJECT OF THE INVENTION AND SUMMARY

The main object of the present invention is to provide a prefillable syringe with safety features which renders it ‘auto-disable’ with user controlled needle retraction.

Another objective of the present invention is to provide an auto-disable prefillable syringe which has simple mechanisms devoid of spring components and which enables administration of the medicine without the possibility of leakage of the fluid.

Yet another objective of the present invention is to provide an auto-disable prefillable syringe that reduces the risk of tampering with the auto-disable mechanism.

A further objective of the present invention is to provide an auto-disable prefillable syringe which is cost-effective.

The auto-disable syringe of the present invention is free from conventional spring mechanisms, making the design simple. The unique features include:
1. the two locking arrangements: the first locking arrangement between the plunger and the plunger extension (i.e. plunger-plunger extension locking arrangement) and the second locking arrangement between the needle holder and the plunger (i.e. needle holder-plunger locking arrangement). These unique locking arrangements help in proper coupling without causing any leakage of the fluid contained in the prefillable syringe
2. the unique design of the barrel tip (i.e. proximal to the needle holder) allows for the needle cap to securely form a seal over the needle and prevent leakage
3. the finger flange extension and/or barrel design prevents the needle holder-plunger locking arrangement from being removed out of the syringe barrel, thereby preventing tampering of the auto-disable mechanism.

### BRIEF DESCRIPTION OF THE DRAWINGS

Preferred forms of the present invention will now be described, by way of non-limiting example only, with reference to the accompanying drawings, in which:

FIG. 1 illustrates a cross sectional view of the prefillable syringe components according to an embodiment of the present invention;

FIG. 2 illustrates a cross sectional view of the main body component of the prefillable syringe depicted in FIG. 1;

FIG. 3 illustrates a cross sectional view of the needle cap of the prefillable syringe depicted in FIG. 1;

FIG. 4 illustrates a cross sectional view of the needle and needle holder of the prefillable syringe depicted in FIG. 1;

FIG. 5 illustrates a cross sectional view of the plunger of the prefillable syringe depicted in FIG. 1;

FIG. 6 illustrates a cross sectional view of the finger flange extension of the prefillable syringe depicted in FIG. 1;

FIG. 7 illustrates a cross sectional view of the plunger extension of the prefillable syringe depicted in FIG. 1;

FIG. 8 is a cross sectional view illustrating a prefillable syringe according to an embodiment of the present invention prior to coupling of the plunger-plunger extension locking arrangement;

FIG. 9 is a cross sectional view illustrating the completed coupling of the plunger-plunger extension locking arrangement;

FIG. 10 is a cross sectional view illustrating the completed coupling of the needle holder-plunger locking arrangement;

FIG. 11 is a cross sectional view illustrating full retraction of the needle into the barrel of the syringe.

FIG. 12 is a cross sectional view illustrating decoupling of the plunger-plunger extension locked arrangement so that the plunger extension can be removed, but the needle holder-plunger locked arrangement is captured within the main body of the prefillable syringe.

### DETAILED DESCRIPTION OF THE INVENTION

### Components of Prefillable Safety Syringe:

In a first aspect, the present invention provides a syringe. The syringe comprises of:
a) a main body consisting of a barrel for holding a medicine or biological for injection and a barrel tip housing the needle holder. The barrel may also have a moulded groove formed in the inside component at the end of the barrel preventing the needle holder-plunger locked arrangement from being removed out of the syringe barrel;
b) a needle cap to securely form a seal over the needle and prevent leakage;
c) a needle holder housed within the body (located at the barrel tip). The said needle holder being configured to hold a needle extending from a first end thereof and including a first locking arrangement accessible from a second end thereof;
d) a plunger located within the barrel such that it forms a seal within the inside surface of the barrel to enable the plunger to force the medicine or biological from the barrel via the needle upon administration. When administering the injection, the plunger is pushed towards the needle holder. The plunger includes a first locking arrangement that is configured to couple with the locking arrangement of the needle holder to lock the plunger to the needle holder (i.e. needle holder-plunger locking arrangement). The plunger also includes a second locking arrangement accessible via a second end thereof;
e) a finger flange extension that is designed to fit over the existing barrel flange. This finger flange extension allows for easier administration of the medicine and may also prevent the needle holder-plunger locked arrangement from being removed out of the syringe barrel.
f) a plunger extension, including locking arrangements, preferably using a lock and key design which can be company and/or product specific that is configured to couple with the second locking arrangement of the plunger to lock the plunger extension to the plunger (i.e. plunger-plunger extension locking arrangement). The plunger extension being configured to enable the plunger to be pushed towards the barrel tip by applying a pushing force to the plunger extension.

### Locking Arrangements:

### Plunger-Plunger Extension Locking Arrangement

By a first action, the locking arrangement between the plunger and the plunger extension should occur. A screwing action is preferable when used to couple the plunger extension to the locking recess of the plunger so as to eliminate the use of direct force onto the medicine or biological and eliminate possibility of leakage of the medicine or biological from the cap.

### Needle Holder-Plunger Locking Arrangement

Then, by a second action, the locking arrangement between the needle holder and the plunger should occur. This second action can occur via a pushing action or application of force by the syringe operator when administering the injection.

The locking arrangement between the needle holder and plunger can include a pin located on the plunger that is received into a correspondingly shaped recess in the mating part of the needle holder. The pin on the plunger can have one or more barbs, or flanges, formed on it that engages with corresponding shaped cavities on the needle holder. Other mechanical engagements may also be used as an alternative.

The design of the syringe precludes the second action occurring before the first action is completed.

All the coupling/insertion actions are in a direction towards the barrel tip.

### Needle Holder and Plunger Locking Arrangement—Tamper Prevention Mechanisms

The syringe can include a capture mechanism arranged to capture and prevent the needle holder-plunger locked arrangement being removed from out of the syringe barrel. Preferably, this capture mechanism may be a separate component, for example, through the use of a finger flange extension that allows for easier administration of the medicine. Additionally or alternatively, this capture mechanism may be formed integrally within the barrel through the use of a moulded groove formed in the inside component at the end of the barrel. Either design feature will prevent access to the needle holder-plunger locked arrangement thereby prevent intentional disassembly of this locked arrangement.

### Needle Mounting and Capping:

The needle holder can include a fitting or feature thereon arranged to receive a hypodermic needle. The needle holder can have a hypodermic needle mounted directly therein, such as a staked needle. The syringe can include a seal located adjacent to the needle holder to prevent leakage of the medicine or biological from the syringe prior to use. This can be in the form of a needle cap to cover the needle (which is mounted to the needle holder) to form a seal to prevent access and leakage of the medicine or biological prior to injection. A unique design of the barrel tip will allow for the said needle cap to securely form a seal over the needle and prevent leakage.

Alternatively, fittings other than a staked needle may also be used. For example, the needle holder can include a luer taper fitting to enable needles to be mounted using either luer-lock or luer-slip fittings. In the case of a luer taper fitting, where the needle has not yet been affixed to the syringe, the syringe can include a plug that is mounted onto the barrel tip to form a seal to prevent access and leakage of the medicine or biological prior to injection.

As used herein, except where the context requires otherwise, the term “comprise” and variations of the term, such as “comprising”, “comprises” and “comprised”, are not intended to exclude further additives, components, integers or steps.

In broad concept, the present invention provides a prefillable syringe in which the plunger extension of the syringe is reversibly coupled to its actuating mechanism during the ordinary process of ejecting the medicine or biological from the syringe. Advantageously, because the plunger extension of the syringe is reversibly coupled to its actuating mechanism, the syringe cannot be used to draw the medicine or biological until the operator is ready to administer the injection.

The first step in the drug injection process is to couple the plunger extension to the plunger via the plunger-plunger extension locking arrangement. Then the cap of the syringe is removed and needle exposed. The injection site is then cleaned in preparation for injection. The needle is then inserted into the injection site and the plunger extension is pushed so that the plunger moves forward, thereby administering the medicine to the patient.

At the end of the stroke, the plunger is locked irreversibly into the needle holder via the needle holder-plunger locking arrangement, providing a visual end-of-dose cue to the syringe operator.

The needle holder remains coupled with the plunger and this locked arrangement can be customised to make the prefillable syringe, company and/or product specific. The needle holder-plunger locked arrangement allows the retraction of the needle into the barrel, preventing the risk of needle stick injuries.

Finally, the plunger extension is pulled away from the barrel such that it reaches the end of the barrel. The use of the finger flange extension and/or a moulded groove formed in the inside component at the end of the barrel allows for decoupling of the plunger-plunger extension locked arrangement so that the plunger extension can be removed and discarded. Either of these unique design features prevents the needle holder-plunger locked arrangement from being removed out of the syringe barrel, thereby preventing access to and tampering of the auto-disable mechanism.

Further aspects of the present invention and further embodiments of the aspects described in the preceding paragraphs will become apparent from the following description, given by way of example and with reference to the accompanying drawings.

### EXAMPLE

### Preferred Embodiment

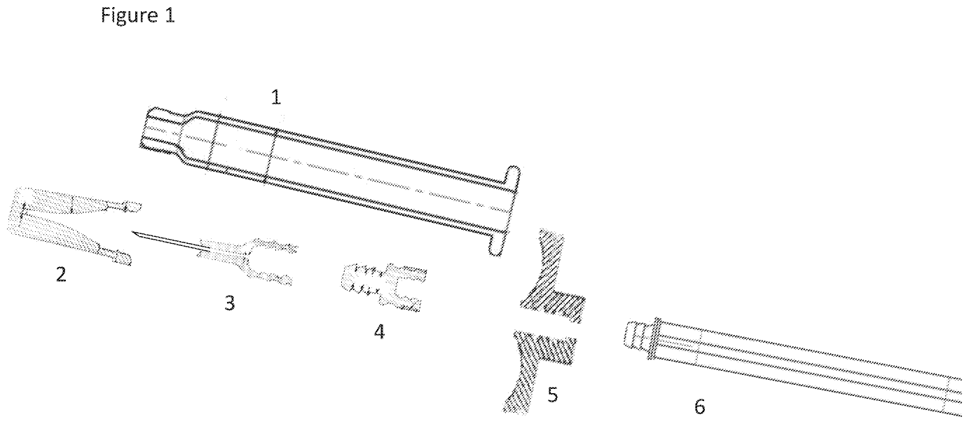

FIG. 1 illustrates a preferred embodiment of a prefillable syringe of the present invention in a cross sectional view. As can be seen, the prefillable syringe includes six main components as follows:

A main body (1);

A needle cap (2);

A needle holder (3);

A plunger (4);

A finger flange extension (5);

A plunger extension (6).

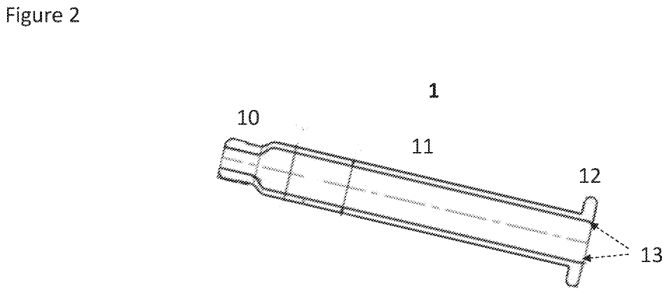

FIG. 2 illustrates the main body of the prefillable syringe (1), including the barrel tip (10) which houses the needle holder and is designed to allow the needle cap to securely form a seal over the needle, the barrel (11) and the barrel flange (12). A moulded groove (13) may be formed in the inside of component at the end of the barrel preventing the needle holder (3)-plunger (4) locked arrangement from being removed out of the barrel of the syringe.

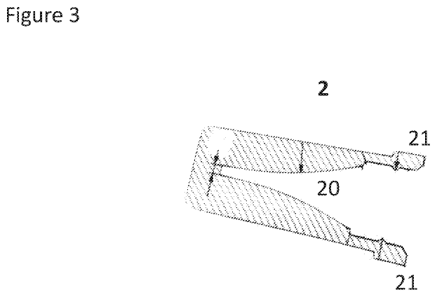

FIG. 3 illustrates the needle cap (2), including the needle sheath (20) and a needle cap seal (21) that couples with the barrel tip (10) as shown in FIG. 2.

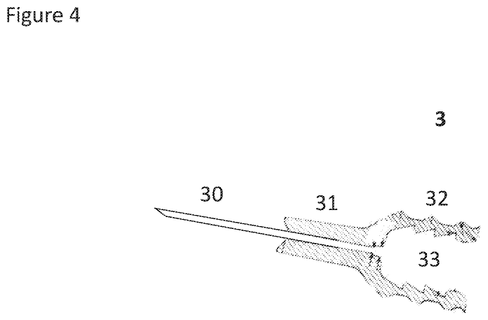

FIG. 4 illustrates a staked needle holder (3) that includes a needle (30), the needle hub (31), the needle holder seal (32) and the locking recess (33).

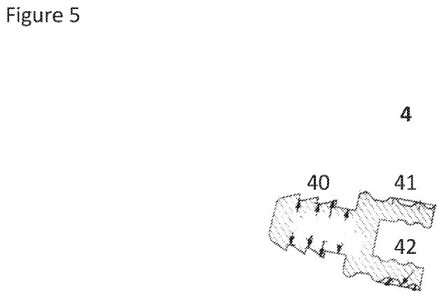

FIG. 5 illustrates the plunger (4), which includes the pin (40), the plunger seal (41) and the locking recess (42).

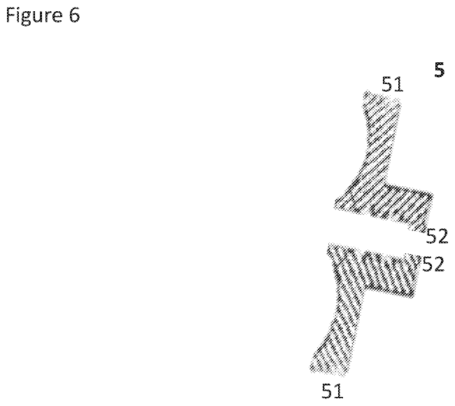

FIG. 6 illustrates the finger flange extension (5), which includes the finger extensions (51) and the grooves (52) that may be used to prevent the needle holder (3)-plunger (4) locked arrangement from being removed out of the barrel.

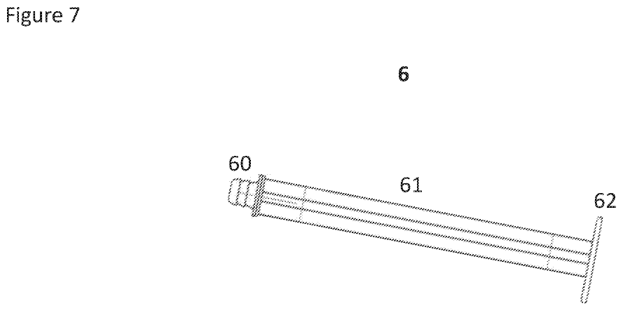

FIG. 7 illustrates the plunger extension (6), including the locking arrangement (60), the plunger rod component (61), and the plunger extension flange (62).

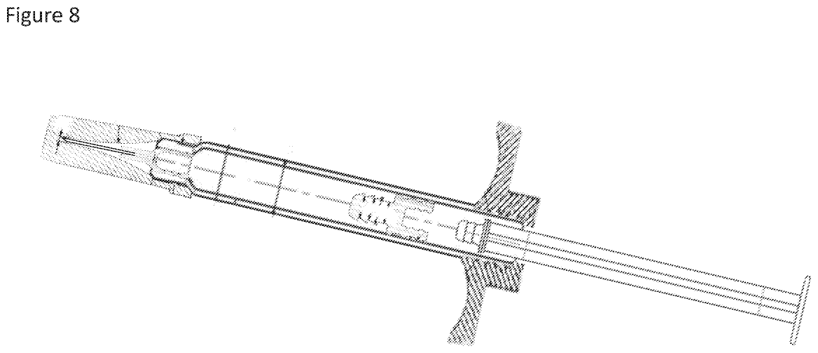

FIG. 8 illustrates the assembled prefillable syringe prior to coupling of the plunger (4)-plunger extension (6) locking arrangement. That is, completion of the locking arrangement between the plunger extension locking arrangement (60) and the plunger locking recess (42). As illustrated in this figure, the needle holder (3) has been secured in the syringe tip (10), with the needle (30) housed in the needle sheath (20). The needle cap (2) is secured to the outer syringe tip (10) via the needle cap seal (21).

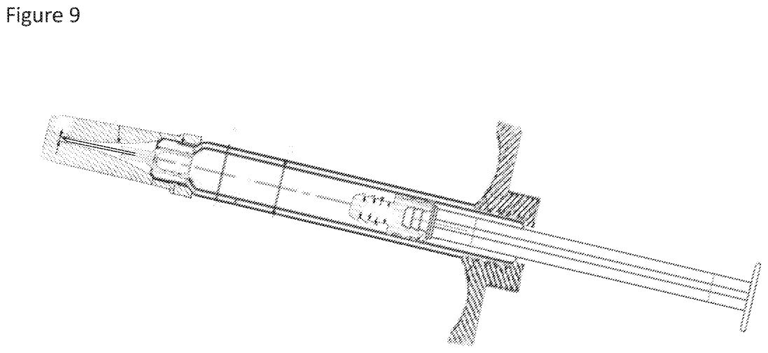

FIG. 9 illustrates the fully assembled prefillable syringe with completion of the plunger (4)-plunger extension (6) locked arrangement. It is preferable that this locking arrangement is via a screwing action between these two components. Once the needle cap (2) is removed, the syringe is now ready for administration of the medicine or biological.

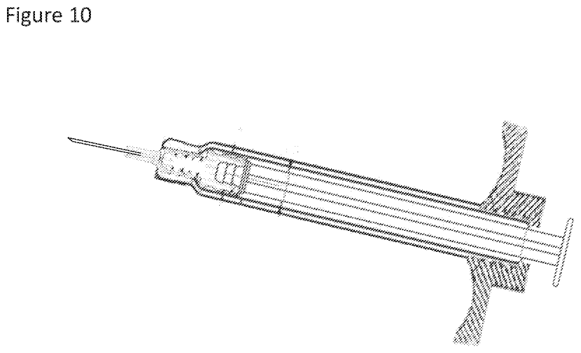

FIG. 10 illustrates completion of administration of the medicine or biological. The plunger pin (40) is now coupled with the needle holder locking recess (33), completing the needle holder (3)-plunger (4) locked arrangement. This provides a visual end-of-dose cue to the syringe operator. The syringe is now auto-disabled.

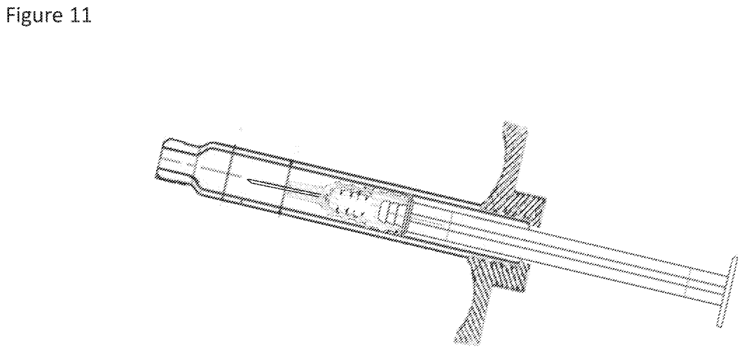

FIG. 11 illustrates retraction of the needle (30) and needle holder (3) into the barrel (11) of the syringe.

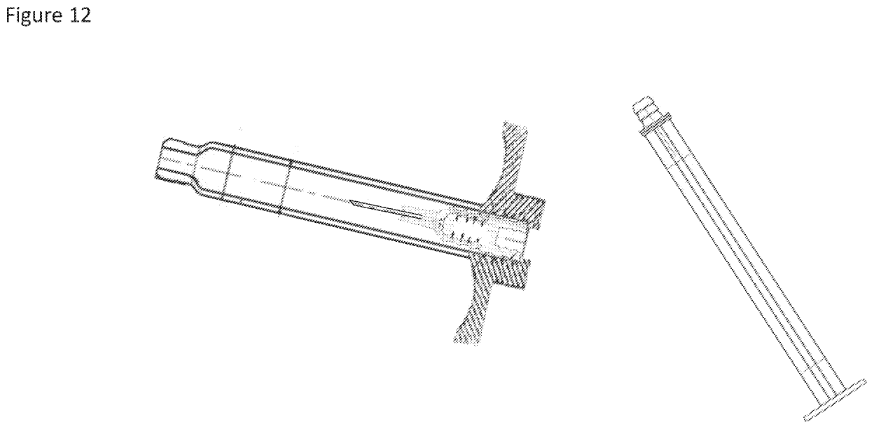

FIG. 12 illustrates the capture mechanism preventing tampering of the auto-disable mechanism. This capture mechanism occurs via the use of the finger flange grooves (52) and/or through the use of a moulded groove formed in the inside component at the end of the barrel. These grooves narrow the diameter at the end of the syringe barrel (11) so that only the plunger extension (6) can be removed. This narrow diameter at the end of the syringe barrel captures the needle holder (3)-plunger (4) locked arrangement and prevents it from being released out of the syringe barrel.

Reference to any prior art in the specification is not, and should not be taken as, an acknowledgment or any form of suggestion that this prior art forms part of the common general knowledge in any other jurisdiction or that this prior art could reasonably be expected to be ascertained, understood and regarded as relevant by a person skilled in the art.

We claim:

1. A prefillable safety syringe comprising of:
a) a main body having a barrel for holding the medicine and a barrel tip for housing a needle holder with a needle (e.g. staked needle) or without a needle (e.g. luer taper),
b) a plunger with a tip capable of forming a locking arrangement with the said needle holder once the medicine has been administered, thus providing a visual end-of-dose cue,
c) a plunger extension with the anterior part capable of forming a reversibly coupled, screwing type of locking arrangement with the posterior end of the plunger,
d) the said plunger extension having a flange located at the posterior part which is configured to enable the plunger to be pushed towards the barrel tip when a pushing force is applied to the plunger extension flange,
e) the said plunger extension allowing for retraction of the needle holder-plunger locking arrangement when a pulling force is applied to the plunger extension flange.

2. The prefillable safety syringe as per claim 1 wherein the needle is either pre-mounted directly onto the needle holder or a needle can be fitted to the needle holder via a luer-lock system.

3. The prefillable safety syringe as per claim 1 wherein the barrel has a means at the posterior end to hold and prevent the needle holder-plunger locking arrangement from being released out of the barrel.

4. The prefillable safety syringe as per claim 3 wherein the means at the posterior end of the barrel is a moulded groove inside the barrel.

5. The prefillable safety syringe as per claim 3 wherein the means at the posterior end of the barrel is a finger flange extension with grooves.

6. The prefillable safety syringe as per claim 1 further comprises a removable needle cap which covers the needle and couples with the barrel tip to form a seal.
